(19) United States
(12) Patent Application Publication
(10) Pub. No.: US 2011/0218493 A1
(43) Pub. Date: Sep. 8, 2011

(54) Tube for Gastrointestinal Tract

(75) Inventors: Susumu Miyasaka, Tokyo (JP); Katsuki Nagata, Tokyo (JP)

(73) Assignee: TYCO HEALTHCARE GROUP LP, Mansfield, MA (US)

(21) Appl. No.: 12/881,295

(22) Filed: Sep. 14, 2010

(30) Foreign Application Priority Data
Sep. 25, 2009 (JP) 2009-219888

(51) Int. Cl. A61M 25/10

(52) U.S. Cl. 604/96.01

(57) ABSTRACT

A tube for use in gastrointestinal tract which allows administration of nutritional, medicinal, or other agent into gastrointestinal tract while preventing detention of digestive juices in esophagus, and which also prevents reflux of gastric contents.

A detention-proof lumen is provided in tube body 12 of gastrointestinal tract tube 10 furnished with tube body 12 equipped with main lumen 12b and expansion lumen, such as balloon lumen 12c, and with an expander such as balloon 15 expanded by delivery of liquid or gas via the lumen provided on outer circumferential surface of tube body 12. Said detention-proof lumen communicates from a part more toward the base than the part where expander is provided on outer circumferential surface of tube body 12 to a part more toward the tip than part where the expander is provided on outer circumferential surface of tube body 12. An inner tube 18 used for liquid supply is provided movably within tube body 12.

### FIELD OF THE DISCLOSURE

The present disclosure concerns a tube used for the gastrointestinal tract [“gastrointestinal tract tube” hereinafter] intended to prevent reflux from the esophagus of gastric contents detained within the gastrointestinal tract in the body, while also administering a nutritional or medicinal agent or the like in the gastrointestinal tract.

### BACKGROUND OF THE DISCLOSURE

Conventionally, a gastrointestinal tract tube is inserted transnasally and placed in the stomach or intestines to administer a nutritional or medicinal agent or the like or to provide iliac treatment. On the gastrointestinal tract tube used in such cases, a balloon is attached to the outer circumferential surface of the tube, and this balloon is expanded to occlude the gastric cardia and esophagus, thereby preventing reflux of the gastric contents from the stomach toward the esophagus.

The body of this gastrointestinal tract tube (iliac tube) is provided with an intestinal content suction hole lumen passing from the base to the tip region of the tube, a gastric content suction hole lumen extending from the base to the intermediate region of the tube, and a gastric balloon lumen extending from the base to the intermediate region of the tube. There are also provided an intestinal content suction hole communicating with the intestinal content suction hole lumen at the tip region of the tube body, and a gastric content suction hole communicating with the gastric content suction hole lumen at the intermediate region. A balloon communicating with the gastric balloon lumen is also provided at the intermediate region of the tube. Consequently, the intestinal contents can be suctioned from the intestinal content suction hole lumen and removed, and the gastric contents can also be suctioned from the gastric content suction hole lumen and removed. The expansion of a balloon at the gastric cardia or esophagus also prevents reflux of the gastric contents from the esophagus.

Nonetheless, though the use of the aforementioned gastrointestinal tract tube prevents reflux of gastric contents from the stomach toward the esophagus, saliva or other such digestive juices accumulate on the esophageal side of the expanded balloon. Such accumulation of digestive juices causes discomfort to the patient.

### SUMMARY OF THE DISCLOSURE

The present disclosure was completed in light of these circumstances and has an object of offering a gastrointestinal tract tube able to prevent reflux of gastric contents from the stomach toward the esophagus, and also able to prevent detention of digestive juices in the esophagus and to administer a nutritional or medicinal agent or the like in the gastrointestinal tract.

To achieve the aforementioned object, the gastrointestinal tract tube pertaining to the present disclosure is characterized structurally in that, in a gastrointestinal tract tube provided with a tube body provided with a main lumen extending from a base part to a tip part and a working expansion lumen extending from the base part toward the tip, and with an expander provided on the outer circumferential surface of the tube body which expands in the transverse direction of the tube body by performance of a predetermined operation via the working expansion lumen; side holes are provided respectively at a part more toward the base than the part where the expander is provided on the outer circumferential surface of the tube body, and at a part more toward the tip than the part where the expander is provided on the outer circumferential surface of the tube body; and a detention-proof lumen communicating with both side holes is provided in the tube wall of the tube body.

### BRIEF DESCRIPTION OF THE DRAWINGS

FIG. 1 is a side view illustrating the gastrointestinal tract tube pertaining to a first Preferred Embodiment of the present disclosure.

FIG. 2 is a cross-sectional view of 2-2 in FIG. 1.

FIG. 3 is a descriptive view illustrating a gastrointestinal tract tube placed in the stomach.

FIG. 4 is a side view illustrating the gastrointestinal tract tube pertaining to a second preferred embodiment of the present disclosure.

FIG. 5 is a side view illustrating the gastrointestinal tract tube pertaining to a third preferred embodiment of the present disclosure.

FIG. 6 is a cross-sectional view of 6-6 in FIG. 5.

FIG. 7 is a descriptive view illustrating the gastrointestinal tract tube in FIG. 6 placed in the stomach.

### DETAILED DESCRIPTION OF THE DISCLOSURE

The gastrointestinal tract tube pertaining to the present disclosure is provided in the tube wall of the tube body with a detention-proof lumen sandwiched at the part where the expander is provided on the outer circumferential surface of the tube body and open at a part more toward the base and more toward the tip than the part where the expander is provided on the outer circumferential surface of the tube body. Consequently, when the tip of the gastrointestinal tract tube is placed within the stomach and the expander is expanded at the gastric cardia in the transverse direction of the tube body, thus occluding the esophagus and preventing reflux of gastric contents from the stomach toward the esophagus, even if digestive juices are detained on the esophageal side of the expander, such digestive juices pass through the detention-proof lumen and enter the stomach. Detention of digestive juices in the esophagus is thus prevented.

In such case, it is preferable that the detention-proof lumen be structured such that digestive juices, etc. cannot move from the stomach toward the esophagus. For example, when digestive juices, etc. attempt to enter the detention-proof lumen at an aperture located more toward the tip of the tube body than the expander in the detention-proof lumen, the provision of a valve occluding the aperture allows passage of digestive juices, etc. only in one direction in the detention-proof lumen. Additionally, administration of a nutritional, medicinal, or other such agent through the aperture is carried out through the main lumen of the tube body. The expander may also be an item expandable in the transverse direction of the tube body by means of a predetermined operation carried out via the working expansion lumen, and other various items may also be used, e.g., a sack-shaped item expanded by entry of a liquid or gas, or one with a structure akin to an umbrella and expanded by use of a wire or like to effect movement of a predetermined part. The tip of the main lumen may also be open at the tip of the tube body and may also be open at a side of the tip of the tube body.

Another structural characteristic of the gastrointestinal tract tube pertaining to the present disclosure is that the main lumen is open at the tip of the tube body, and an inner tube used for supply of a liquid and extending from the base of the tube body along the inside of the main lumen and projecting from the opening is provided in the tube body.

In such case, administration of a nutritional, medicinal, or other such agent to the stomach or intestine is carried out not by means of the main lumen of the tube body, but by means of the inner tube. Captain is procedure, once the tip of the tube body has arrived at a location just inside the stomach, the expander is expanded and thereby secured to the tube body, and the inner tube alone can be inserted into the intestinal side of the stomach or the intestine to administer a nutritional, medicinal, or other such agent. In other words, the expander is secured to the gastric cardia and prevents reflux of stomach contents from the stomach toward the esophagus, and in this state, a nutritional, medicinal, or other such agent can be administered to the stomach or intestine by advancing the tip of the inner tube to a desired location and inserting the same in the intestinal side of the stomach or the intestine.

Yet another structural characteristic of the gastrointestinal tract tube pertaining to the present disclosure is that suction side holes are provided at a part more toward the tip than the part where the expander is provided on the outer circumferential surface of the tube body, and a suction lumen communicating from the base of the tube body to the suction side holes is provided in the tube wall of the tube body. The aperture of the suction lumen is thereby positioned at the tip part of the tube body, and consequently the inside of the stomach can be suctioned or vacuumed as needed.

Yet another structural characteristic of the gastrointestinal tract tube pertaining to the present disclosure is that the expander comprises a balloon, and performance of a predetermined operation via the working expansion lumen comprises an operation which delivers a liquid or gas into the balloon. The esophagus or gastric cardia can thereby be occluded reliably by a simple structure. The shape of the balloon when expanded may also be spherical, barrel shaped, or various other shapes.

### DETAILED DESCRIPTION OF THE DRAWINGS

In the following examples, the following symbols are used:

10, 20, 30) gastrointestinal tract tube; 11, 31) outer tube; 12, 22, 32) tube body; 12b, 32b) main lumen; 12c, 32c) balloon lumen; 15, 35) balloon; 16a, 16b) inlet side hole; (Text refers to “Inlet holes 16a, 17a” and “outlet holes 16b, 17b.”) outlet side hole; 37) suction side hole; 18) inner tube; 25) expansion dome; 32d) suction lumen.

### Preferred Embodiment 1

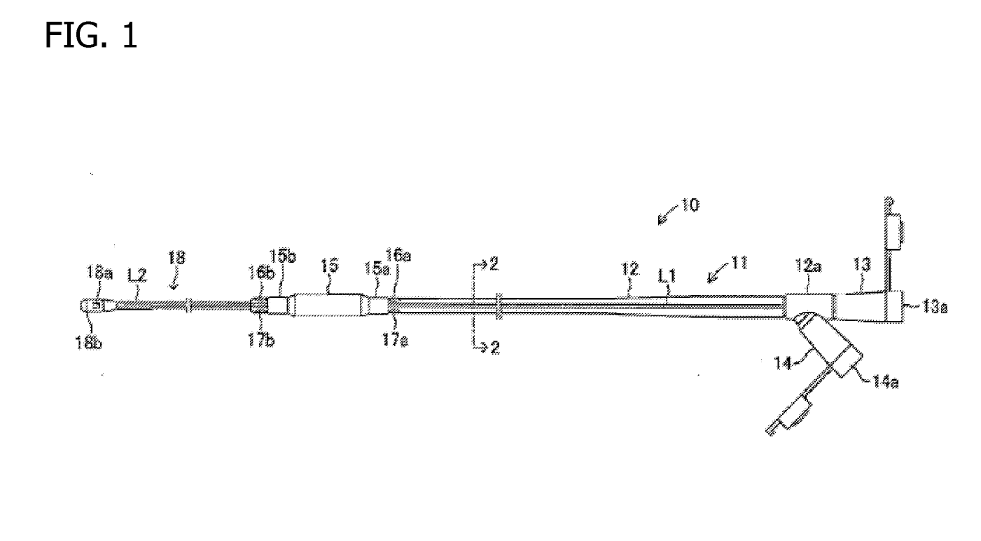

A first preferred embodiment of the gastrointestinal tract tube pertaining to the present disclosure is described in detail using figures. FIG. 1 illustrates a gastrointestinal tract tube 10 pertaining to the Preferred Embodiment, and this gastrointestinal tract tube 10 is used to administer a nutritional agent or a medicinal agent or the like transnasally to the stomach S or intestine I (cf. FIG. 3 and FIG. 7) of a patient. The gastrointestinal tract tube 10 comprises an outer tube 11, and an inner tube 18 provided movably within the outer tube 11. The outer tube 11 is provided with a tube body 12 comprising a long, slender resin tube; branching tubes 13, 14 branching from the base end 12a of the tube body 12; and a balloon 15 provided near the tip of the tube body 12 and serving as the expansion area pertaining to the present disclosure.

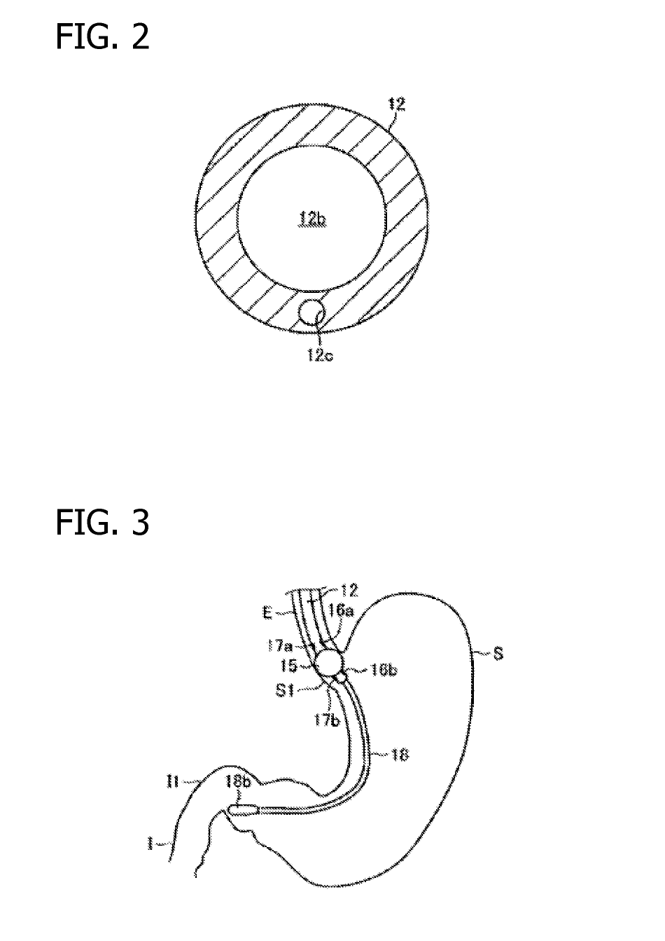

As shown in FIG. 2, a large-diameter main lumen 12b is formed at the interior center of the tube body 12, and a small-diameter balloon lumen 12c is formed laterally (lower portion of FIG. 2) to the main lumen 12b in the tube wall of the tube body 12. The base end 12a of the tube body 12 is provided with two flow paths communicating with the main lumen 12b and the balloon lumen12c. The main lumen 12b extends from the flow path formed at the center of the base end 12a to the tip of the tube body 12. The base end of the main lumen 12b also communicates via the flow path within the base end 12a directly to a branching tube 13. A connector 13a is formed at the back end of the branching tube 13, and other devices or tubes or the like can be connected via the connector 13a to the branching tube 13 as needed.

The balloon lumen12c extends from one more flow path formed in the base end 12a toward the tip of the tube body 12 and communicates with the balloon 15. The base end of the balloon lumen12c communicates via the flow path within the base end 12a to a branching tube 14. The branching tube 14 comprises a tube body of approximately the same diameter as the aforementioned branching tube 13a, inclines from the side of the base end 12a, and extends rearward obliquely. A connector 14a is formed at the back end of the branching tube 14. A syringe or other such supply device for delivering water or air to the balloon 15 is connected to this connector 14a.

Operation of the supply device and delivery of water or air into the balloon 15 expands the balloon 15 to an appropriate state. The balloon 15 comprises an elastic, soft resin film, and its back end 15a and tip end 15b are each secured to the outer circumferential surface of the tube body 12. During expansion and contraction, as shown in FIG. 1, the balloon assumes a state of close contact with the outer circumferential surface of the tube body 12, but when water or air is supplied, the balloon expands and assumes a spherical, cylindrical, spindle, or other such shape.

Inlet side holes 16a, 17a are formed at the base end of the area where the balloon 15 is provided on the outer circumferential surface of the tube body 12 and are formed in a manner maintaining 180° spacing peripherally. Outlet side holes 16b, 17b are formed at the tip end of the area where the balloon 15 is provided on the outer circumferential surface of the tube body 12 and are formed in a manner maintaining 180° spacing peripherally. One inlet side hole 16a and outlet hole 16b are brought into communication by a detention-preventing lumen (not illustrated) formed in the tube wall of the tube body 12, and the other inlet side hole 17a and outlet hole 17b are brought into communication by a detention-preventing lumen (not illustrated) formed in the tube wall of the tube body 12. A contrast imaging line L1 comprising an X-ray opaque agent is also formed in the tube body 12 from the tip onward to the base end.

In the inner tube 18, a tip-side portion is projected from the tip end of the outer tube 11 and disposed within the outer tube 11, and the length of projection from the tip end of the outer tube 11 can be changed by advancing or withdrawing the base end portion relative to the outer tube 11. A lumen (not illustrated) extending from the base end to the tip is also provided in the inner tube 18, and at the base end thereof, a supply device for supplying a nutritional agent, drug solution, or other such agent is connected to the lumen.

A spindle 18b provided with a side hole 18a for nutrition supply is attached to the tip of the inner tube 18. A hole communicating with the lumen of the inner tube 18 is formed in the interior of the spindle 18b, and two side holes 18a for nutrition supply are formed extending from the vicinity of the occluded tip (of the spindle) perpendicularly, and in respectively opposite directions. A contrast imaging line L2 comprising an X-ray opaque agent is also formed in the body of the inner tube 18 from the tip onward to the base end.

Through this structure, when a nutritional, medicinal or other such agent is administered to the stomach S or intestine I of a patient using the gastrointestinal tract tube 10, a stylet (not illustrated) is first inserted into the space within the inner tube 18, and the adapter of the stylet is secured to the base of the inner tube 18. Next, a gastrointestinal tract tube 10 in the state shown in FIG. 1 is inserted together with the stylet into the nose from the side of the inner tube 18 spindle 18b. Then, as shown in FIG. 3, when the outer tube 11 balloon 15 has reached the gastric cardia S1, air is supplied from a supply apparatus via a branching tube 14 and balloon lumen 12c into the balloon 15, and the balloon 15 is expanded. The balloon 15 thus occludes the gastric cardia S1 and thereby blocks both the stomach S and esophagus E, and the tip side of the outer tube 11 is secured by means of the balloon 15.

Next, the inner tube 18 is moved with respect to the outer tube 11, and the spindle 18b at its tip reaches the intestine side region of the stomach S or the pylorus I1, which is the boundary between the stomach S and the intestine I. Such movement of the tip of the inner tube 18 is effected smoothly by the fact the spindle 18b provided at the tip of the inner tube 18 has a predetermined weight. An adjustably-positioned stopper (not illustrated) is also attached to the outer circumferential surface of the tube body 12, and by means of this stopper, only an appropriate length of the gastrointestinal tract tube 10 is inserted. After insertion, the body of the patient is X-rayed to image and thereby confirm the position of the contrast imaging line L1 formed in the tube body 12 and the contrast imaging line L2 formed in the body of the inner tube 18.

At this point, the stylet is withdrawn from the interior of the inner tube 18, a supply apparatus is connected to the base of the inner tube 18, and a nutritional, medicinal, or other such agent is supplied from the delivery apparatus via the lumen of the inner tube 18 to the stomach S or intestine I. At such time, the expanded balloon 15 prevents the contents of the stomach S from refluxing toward the esophagus E. Digestive juices or other such fluids also accumulate at the circumferential surface of the expanded balloon 15 on the esophagus E side, but such fluids enter each detention-proofed lumen from inlet side holes 16a, 17a on the esophagus E side of the outer tube 11, pass through each detention-proofed lumen, and flow into the stomach S from outlet holes 16b, 17b on the stomach S side.

Thus, in the gastrointestinal tract tube 10 pertaining to the present preferred embodiment, inlet side holes 16a, 17a are provided on the outer circumferential surface of the tube body 12 more toward the base than the area where the balloon 15 is provided, and outlet holes 16b, 17b are provided on the outer circumferential surface of the tube body 12 more toward the tip than the area where the balloon 15 is provided. In addition, one set of inlet side hole 16a and outlet hole 16b and another set of inlet side hole 17a and outlet hole 17b communicate respectively through the detention-proof lumens formed in the wall of the tube body 12, and the inner tube 18 is provided in the outer tube 11 movably with respect to the outer tube 11.

Consequently, positioning of the tip of the gastrointestinal tract tube 10 within the stomach S and expanding of the balloon 15 at the gastric cardia S1 prevents the contents of the stomach S from refluxing toward the esophagus E, and at such time, digestive juices accumulating on the esophagus E side of the balloon 15 also pass through the two detention-proof lumens and enter the stomach S. As a result, detention of digestive juices in the esophagus E is prevented. And because administration of a nutritional, medicinal, or other such agent to the stomach S or intestine I is also effected by means of the inner tube 18 made movable with respect to the outer tube 11, a nutritional, medicinal, or other such agent can be administered by inserting just the inner tube 18 to a desired position toward the intestinal part of the stomach S or in the intestine I.

### Preferred Embodiment 2

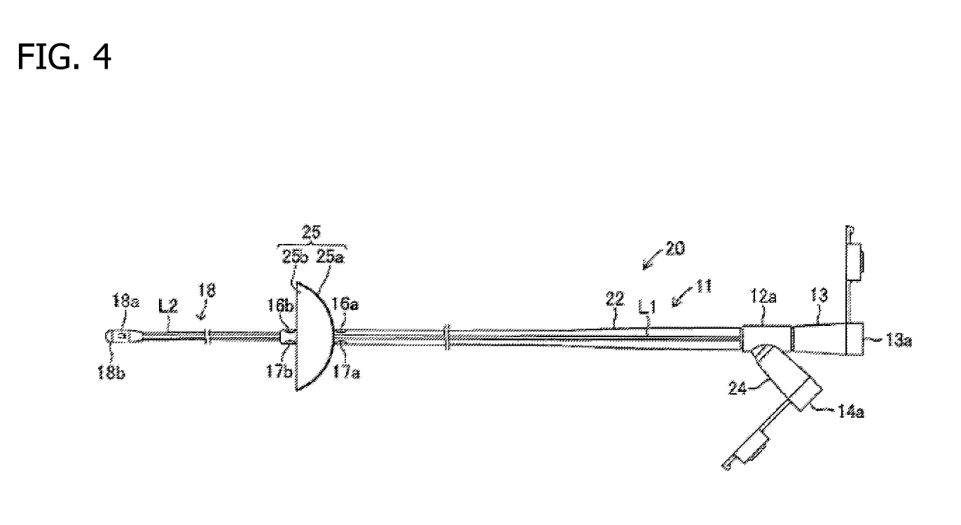

FIG. 4 illustrates a gastrointestinal tract tube 20 pertaining to a second preferred embodiment of the present disclosure. In said gastrointestinal tract tube 20, rather than a balloon 15, a dome-shaped expansion dome 25 is provided as an expanding part. Said expansion dome 25 comprises a plurality of structural parts 25a made from a somewhat rigid resin, a dome-shaped sack 25b made from an elastic, flexible resin film affixed to said structural part 25a, and a sliding part (not illustrated) made movable along the outer circumferential surface of a tube body 22. The center of the structural parts 25a and dome-shaped sack 25b respectively is secured to the outer circumferential surface of the tube body 22, and the sliding part is connected to a wire (not illustrated) extending from a branching tube 24 through a dome lumen (identical to balloon lumen 12c illustrated in FIG. 2) provided in the tube body 22 and serving as a working expansion lumen pertaining to the present disclosure.

When the wire is pulled and moves the sliding part toward the base of the tube body 22, the center portion of the structural parts 25a is pressed, and the structural parts 25a and dome-shaped sack 25b are pressed and spread into a dome shape. At such time, the dome-shaped sack 25b elongates into a dome shape. When the wire is pressed and moves the sliding part toward the tip of the tube body 22, contraction of the dome-shaped sack 25b impels the structural parts 25a toward the outer circumferential surface of the tube body 22, and said parts assume a rod shape in close contact with the outer circumferential surface of the tube body 22. At such time, the contraction of the dome-shaped sack 25b prevents the tip of each structural part 25a from opening unexpectedly, and a separate member or mechanism intended to prevent the tip of each structural part 25a from opening need not be provided.

The structure of other parts of the gastrointestinal tract tube 20 is the same as that of the gastrointestinal tract tube 10 described above. Consequently, identical parts are designated by identical symbols, and a description is omitted. Because the gastrointestinal tract tube 20 pertaining to the present preferred embodiment makes the stomach S surface side of the expansion dome 25 concave, reflux of stomach S contents toward the esophagus E is prevented even more reliably. The operation and effect of this gastrointestinal tract tube 20 are in other respects similar to those of the gastrointestinal tract tube 10 described above. As a variation of the gastrointestinal tract tube 20 pertaining to Preferred Embodiment 2, the orientation of the expansion dome 25 can also be reversed. The stomach S surface of the expansion dome 25 is thereby made convex and occludes the gastric cardia S1 in a covering fashion, and reflux of stomach S contents toward the esophagus E is prevented. Likewise, resistance of the expansion dome 25 during insertion of the gastrointestinal tract tube 20 is also prevented.

### Preferred Embodiment 3

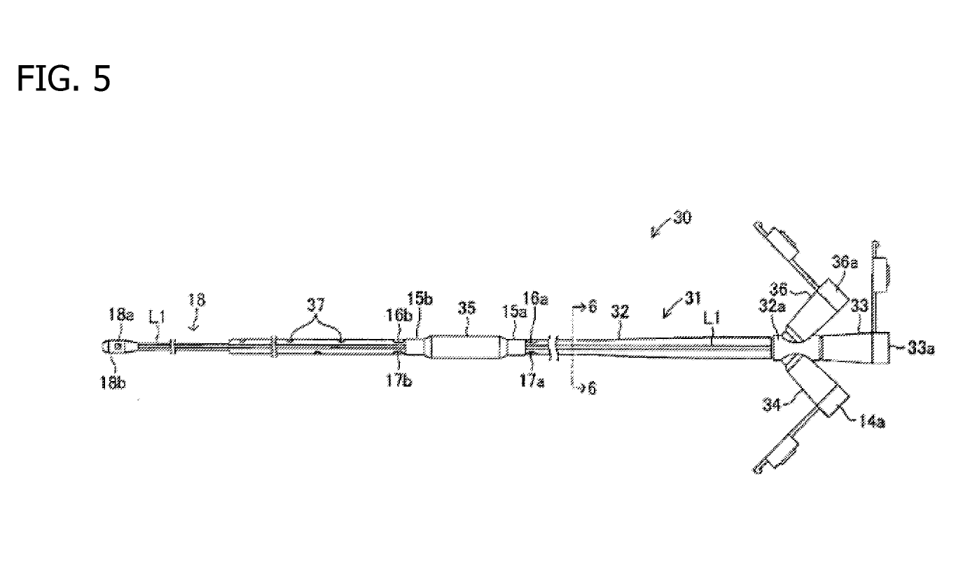

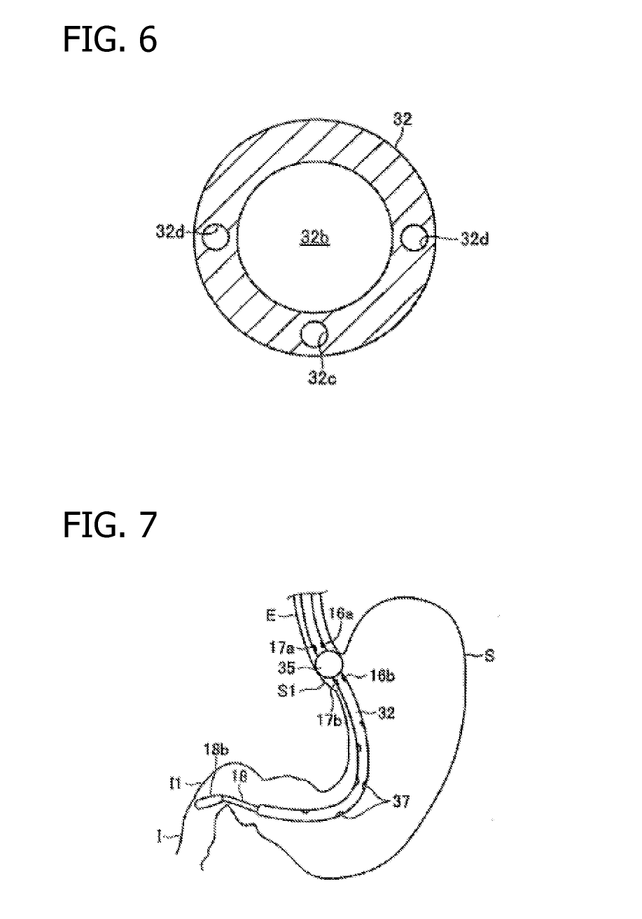

FIG. 5 illustrates a gastrointestinal tract tube 30 pertaining to a third preferred embodiment of the present disclosure. In said gastrointestinal tract tube 30, an outer tube 31 is provided with a tube body 32 comprising a long, slender tube; three branching tubes 33, 34, 36 made from resin and branching from the base 32a of the tube body 32; and a balloon 35 provided from the tip of the tube body 32 toward the base at an area separated only by a predetermined distance. As shown in FIG. 6, a large-diameter main lumen 32b is formed at the interior center of the tube body 32, and a balloon lumen 32c and two suction lumens 32d are formed at the tube wall of the tube body 32.

The base 32a of the tube body 32 is provided with three flow paths communicating with the main lumen 32b, balloon lumen 32c, and suction lumens 32d. The main lumen 32b communicates with a branching tube 33 via a central flow path within the base 32a, and the balloon lumen 32c communicates with another branching tube 34 via another flow path formed in the base 32a. The suction lumens 32d communicate with another branching tube 36 via the remaining other flow path formed in the base 32a. One branching tube 33 extends straight from the base 32a, and the other branching tubes 34, 36 incline respectively from both sides of the base 32a and extend obliquely backward in opposite directions. A suction device or other such suction apparatus is connected to the back end of one branching tube 36 via a connector 36a.

The part of the tube body 32 more toward the tip than the part where the balloon 35 is provided is made longer than the aforementioned tip part of the tube body 12 of the gastrointestinal tract tube 10, and on its outer circumferential surface there open a plurality of side holes 37 for suction which communicate with the suction lumens 32d. In other words, said outer tube 31 is one formed such that a branching tube 36 and suction lumens 32d are provided in the aforementioned outer tube 11 of the gastrointestinal tract tube 10, and in which there is one additional flow path in the base 32a which extends along the tip part of the tube body 32, and in said part there are formed a plurality of suction side holes 37 which communicate with suction lumens 32d. The structure of other parts of said gastrointestinal tract tube 30 is identical to that of the gastrointestinal tract tube 10 described above. Consequently, identical parts are designated by identical symbols, and a description is omitted.

When this gastrointestinal tract tube 30 is used to administer a nutritional, medicinal, or other such agent to the stomach S or intestine I of a patient, as when the gastrointestinal tract tube 10 described above is used, the gastrointestinal tract tube 30 is placed in the stomach S. In such instance, as shown in FIG. 7, the tip part of the tube body 32 is positioned at the intestinal side of the stomach S. If, for this purpose, the pressure within the stomach S must be reduced or liquids etc. in the stomach S must be removed in order to prevent reflux of the stomach S contents toward the esophagus E, a suction apparatus can be operated to suction the interior of the stomach S. Liquids or gases within the stomach S will thereby enter the suction lumens 32d from the suction side holes 37 and will be suctioned from the branching tube 36 into the suction apparatus. The operation and effect of the gastrointestinal tract tube 30 are in other respects similar to those of the gastrointestinal tract tube 10 described above.

As another preferred embodiment, the expansion dome 25 in the second preferred embodiment described above can be used in lieu of the balloon 35 of the gastrointestinal tract tube 30 described above. In such instance as well, an operation and effect equivalent to those of the gastrointestinal tract tube 30 are obtained. As yet another preferred embodiment, an item wherein the inner tube 18 is removed from any of the gastrointestinal tract tubes 10, 20, 30 described above can be used as a gastrointestinal tract tube. In such instance, a suction device is connected to a branching tube 13, 33 connector 13a, 33a, and a nutritional, medicinal, or other such agent is supplied to the stomach S or intestine I from the main lumen 12b, 32b of the outer tube 11, 31.

The therapeutic tube pertaining to the present disclosure is also not limited to the respective preferred embodiments described above and can be implemented with changes as appropriate. For example, in each of the preferred embodiments described above, the outlet side holes 16b, 17b are established as holes opening onto the outer circumferential surface of the tube body 12, 32, but a valve can be provided in said outlet side holes 16b, 17b to prevent movement of digestive juices or the like from the stomach S toward the esophagus E in a detention-proof lumen. For example, a tab able to cover the aperture of the outlet side holes 16b, 17b can be secured to a portion of the aperture edges of the outlet side holes 16b, 17b, and movement of digestive juices, etc. from the stomach S toward the esophagus E can be prevented by a design such that said tab swings at the inside of the stomach S but occludes the outlet side holes 16b, 17b when pressed against the inside of the outlet side holes 16b, 17b.

Movement of digestive juices, etc. from the stomach S toward the esophagus E can also be prevented by providing two tabs at the apertures of the outlet side holes 16b, 17b such that the tips of these two taps protrude toward the inside of the stomach S and are impelled by each other. In such an instance, when digestive juices, etc. move from the esophagus E toward the stomach 5, the two tabs open, and when the digestive juices, etc. attempt to move from the stomach S toward the esophagus E, the two tabs close. The outlet side holes 16b, 17b can also be given a slit shape to allow passage of digestive juices, etc. from a detention-proof lumen into the stomach S but prevent passage from the stomach S into a detention-proof lumen. An expander is also not limited to a balloon 15, 35 or an expansion dome 25 and may also be one which expands transversely in the tube body 12, 22, 32 when a predetermined operation is carried out.

What is claimed is:

1. A gastrointestinal tract tube provided with a tube body provided with a main lumen extending from a base part to a tip part and a lumen for expansion oriented from the base part toward the tip, and with an expander provided on the outer circumferential surface of the tube body which is expanded in the transverse direction of the tube body by the lumen for expansion; characterized in that side holes are provided respectively at a part more toward the base than the part where the expander is provided on the outer circumferential surface of the tube body, and at a part more toward the tip than the part where the expander is provided on the outer circumferential surface of the tube body; and a detention-proof lumen communicating with the side holes is provided in the tube wall of the tube body.

2. The gastrointestinal tract tube according to claim 1, or claim 2, wherein the main lumen is open at the tip of the tube body, and an inner tube used for supply of a liquid and extending from the base of the tube body along the inside of the main lumen and projecting from the opening is provided in the tube body.

3. The gastrointestinal tract tube according to any one of claim 1 through claim 3, wherein suction side holes are provided at a part more toward the tip than the part where the expander is provided on the outer circumferential surface of the tube body, and a suction lumen communicating from the base of the tube body to the suction side holes is provided in the tube wall of the tube body.

4. The gastrointestinal tract tube according to claim 1, wherein the expander comprises a balloon, and the lumen for expansion delivers a liquid or gas into the balloon.
